(19) United States
(12) Patent Application Publication
(10) Pub. No.: US 2009/0095304 A1
(43) Pub. Date: Apr. 16, 2009

(54) Vaginal pessary for the management of stress incontinence

(76) Inventors: Sharon Ann Richardson, Tualatin, OR (US); R.E. Clenton Richardson, Tualatin, OR (US)

Correspondence Address: SHARON ANN RICHARDSON, 4585 SW TRAIL ROAD, TUALATIN, OR, 97062, US

(21) Appl. No.: 11/907,542

(22) Filed: Oct. 15, 2007

(51) Int. Cl. A61F 6/08

(52) U.S. Cl. 128/834

(57) ABSTRACT

The invention is an intra-vaginal pessary device constructed of bio-compatible, medical grade, non-latex silicone, to manage and diagnose mild to moderate stress incontinence in women by providing a user-friendly, non-surgical, occasional, non-invasive option for mid-urethral support. The invention is an approximately oval, non-absorbent device with an embedded pull-string for removal when not in use. The pessary is self-inserted into the vagina by a woman, using the same technique she would use if she were inserting a tampon. The pressure the pessary provides against the vaginal wall has the effect of closing the urethra and thereby preventing urine leakage.

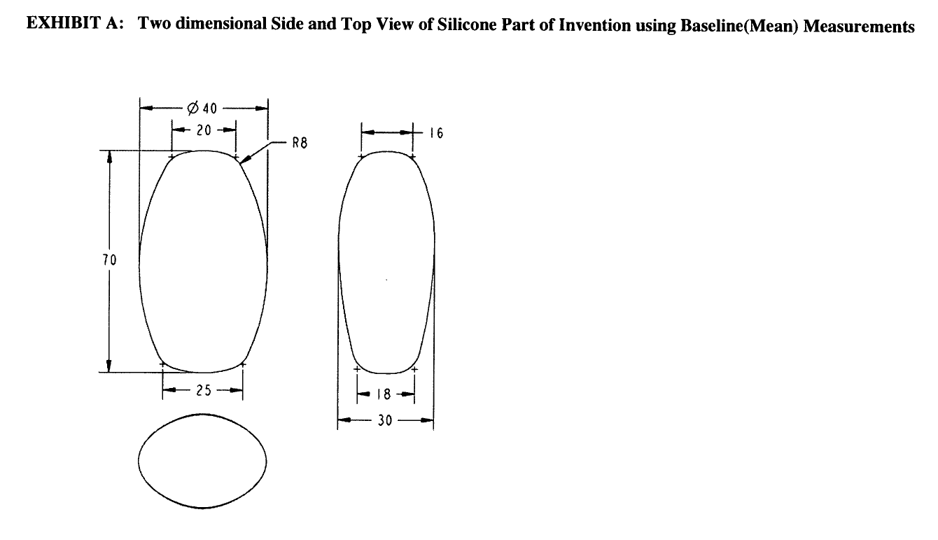

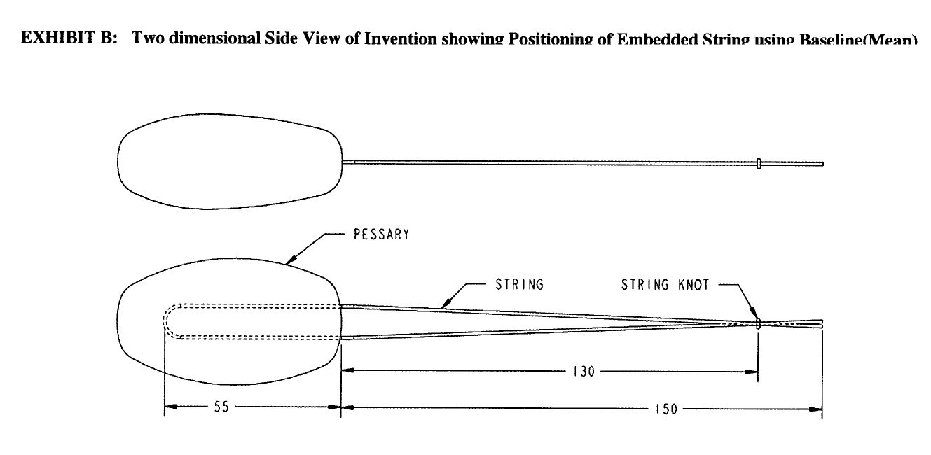

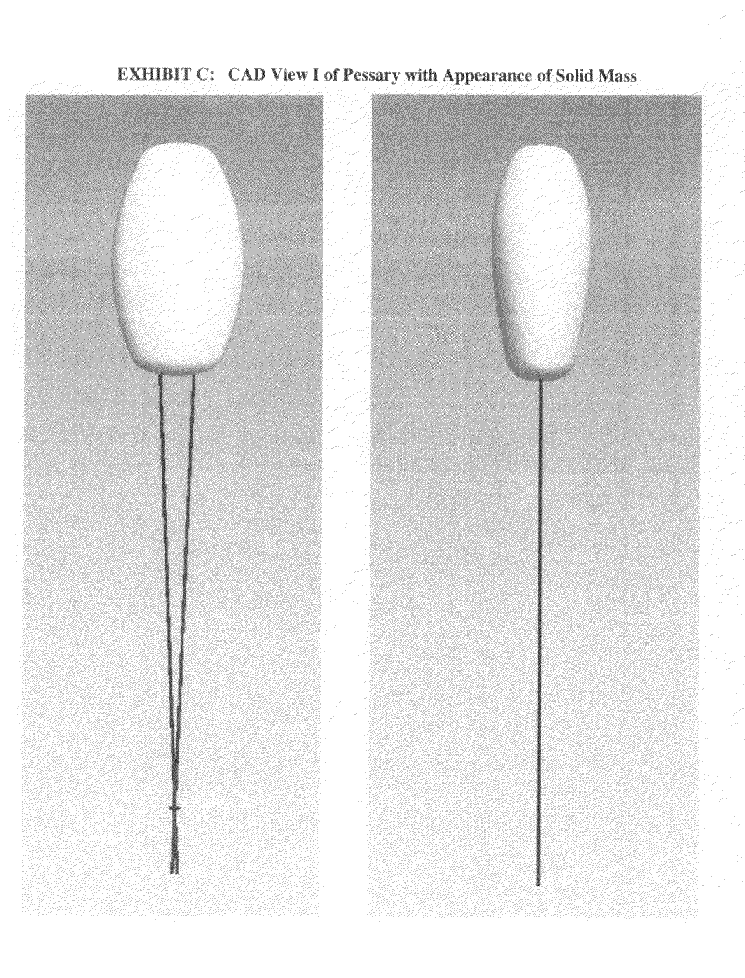

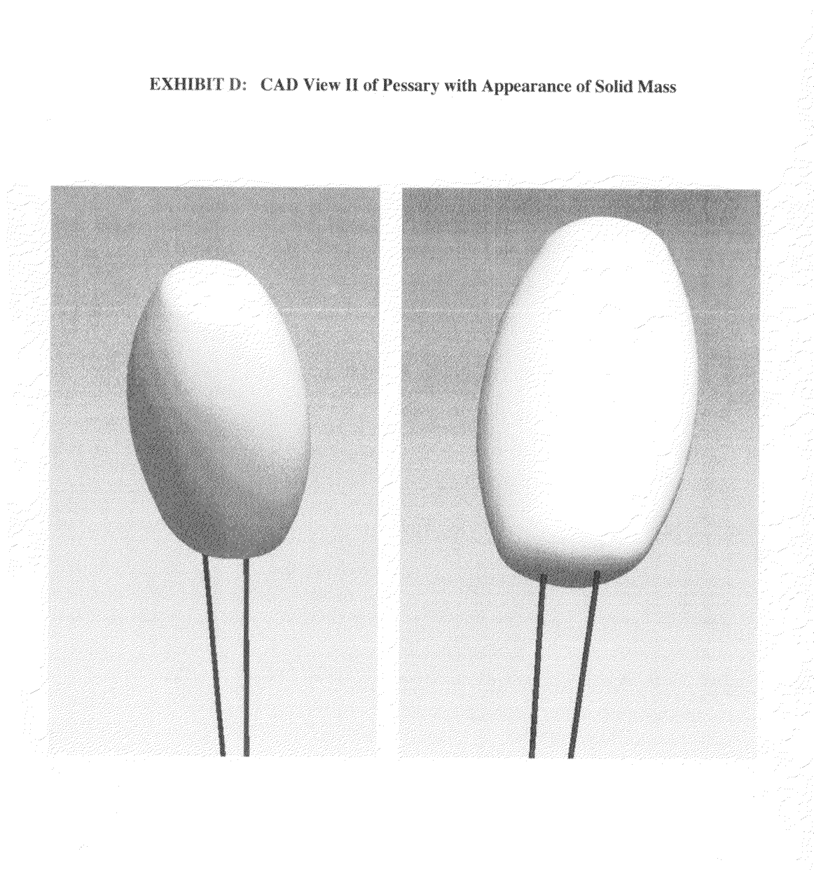

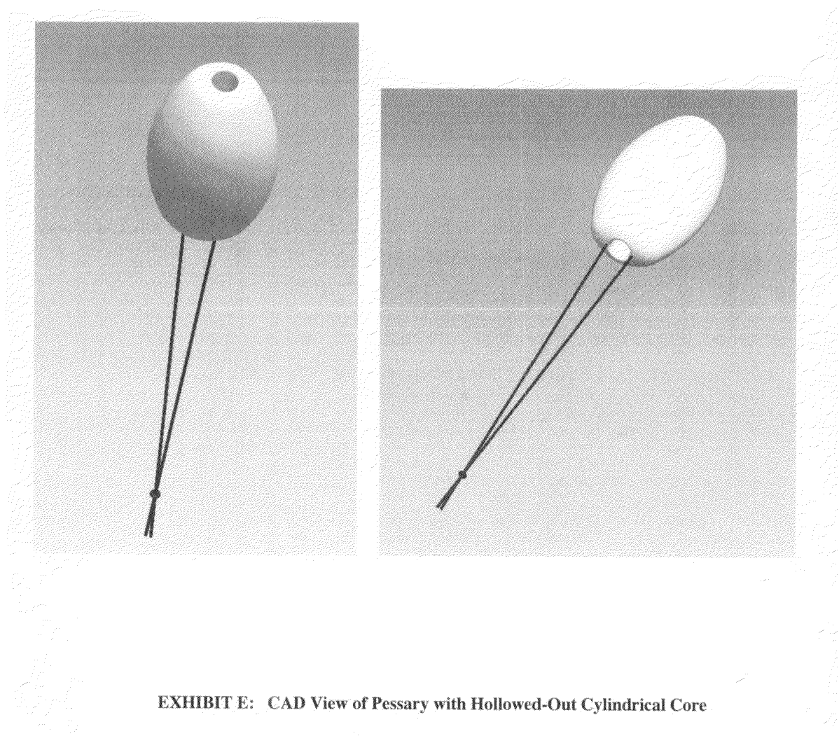

The applicants have no knowledge of relevant cross references from co-pending applications, as required by 35 USC §§120, 121 or 365(C) and this invention was not supported by federally sponsored research and development.

### BACKGROUND OF THE INVENTION

Suggested US Class: 128/884, 600/29, 128/834, 604/904

Field of Search: 128, 600, 105, 523, 520, 884, 885, 869, 834, 604, 904

### US Patent Documents:

| | | |
|---|---|---|
| 3,705,575 | December 1972 | Edwards |
| 4,019,498 | April 1977 | Hawtrey et al. |
| 4,307,716 | December 1981 | Davis |
| 4,920,986 | May 1990 | Biswas |
| 5,224,494 | July 1993 | Enhorning |
| 6,158,435 | December 2000 | Dorsey |

Despite the medical advances and numerous inventions in the classes noted above, women who are afflicted with stress urinary incontinence are still very limited in terms of their choices. Most alternatives are a) invasive, requiring surgery; b) expensive, requiring lengthy consultations and complex fittings with expert physicians; or c) antiquated, with tremendous potential for embarrassment. A simple, straightforward, intuitive, and affordable alternative that women can potentially choose for themselves does not currently exist in the market. Our invention is designed to fill this critical need.

### SUMMARY OF THE INVENTION

Our invention is a pessary, with utility of temporarily alleviating mild to moderate stress incontinence in women by applying pressure onto the urethra from within the vagina. The simplicity of the design which more closely resembles female anatomy and the fact that it is insertable in much the same way as a tampon makes it more likely that women will use it correctly and be able to ascertain fairly quickly whether their particular form of incontinence is manageable by such a temporary method or whether surgery and/or a more permanent and potentially invasive method is required.

The prevalence of incontinence in the United States is widespread, especially in light of our aging population. We have anecdotal evidence showing that, given the cost of the pessaries in the market, coupled with the social stigma attached to incontinence, women with this problem find themselves having to rely on absorbent adult protection products such as sanitary pads and adult diapers.

Regular check-ups with a physician are very important and our invention in no way alleviates this critical need. In fact, our partners, who will help us determine via patient testing whether this product is ready for the point of sale, are physicians. However, mild to moderate stress incontinence is manageable, and with a product that enables purchase at retail in much the same way as a condom or tampon is purchased, the invention offers a dual benefit: 1) by alleviating the problem of urinary incontinence and 2) by raising the social stigma of the condition itself.

The exhibit in Appendix I on page 12 provides an overview of pessary products currently in the market, that are currently being prescribed and fitted through physicians' offices.

### DETAILED DESCRIPTION OF THE INVENTION

Our invention is an intra-vaginal pessary device to manage and diagnose mild to moderate stress incontinence in women by providing a non-surgical, non-invasive option for mid-urethral support.

The pessary device is constructed of bio-compatible, medical grade, non-latex silicone. Medical grade silicone of approximately 50 durometer is currently under consideration, but note that this durometer may be subject to change for reasons of comfort, weight of the invention, and durability.

High-temperature molding and tooling techniques are used to manufacture an approximately oval, non-absorbent device that somewhat resembles a loosened tampon. It has a pull-string embedded in the molded silicone to remove it when not in use. The pessary is self-inserted into the vagina by a woman, using the same technique she would use if she were inserting a tampon.

The pressure the pessary provides against the vaginal wall has the effect of closing the urethra and thereby preventing urine leakage.

The string itself is a braided suture that naturally resists adherence so that cleaning the pessary and bacterial accumulation resistant to this cleaning will not be an issue.

The invention may be used either as a pessary of solid mass or one with a hollowed-out core that is roughly cylindrical to enable the passage of vaginal fluids through this core and/or lessen the weight of the pessary (Exhibit E).

Additionally, we will vary the material to accommodate differences in usage preferences and user price sensitivity, so that at least 3 embodiments of the device may exist: 1) Single use, disposable usage 2) Short-term disposable use, classified as up to 20-30 days from first use, that allows the user to wear the pessary up to 8 hours at a time and requires cleaning between uses. 3) Longer-term use, classified as giving the user greater than 30 days from first use but no more than 120 days in total, that allows the user to wear the pessary up to 8 hours at a time and requires cleaning between uses.

### DESCRIPTION OF DRAWINGS

EXHIBIT A: Two dimensional Side and Top View of Silicone Part of Invention using Baseline (Mean) Measurements.
The two-dimensional drawing in Exhibit A uses baseline or mean measurements and two different views (side and top) to illustrate the silicone portion of the invention.
EXHIBIT B: Two dimensional Side View of Invention showing Positioning of Embedded String using Baseline (Mean) Measurements.
The two-dimensional drawing in Exhibit B uses baseline or mean measurements to illustrate the positioning and construction of the embedded string described in the invention.
EXHIBIT C: CAD View I of Pessary with Appearance of Solid Mass
First set of CAD views to illustrate the pessary with the external appearance of a solid mass.
EXHIBIT D: CAD View I of Pessary with Appearance of Solid Mass
A second set of CAD views to illustrate the pessary with the appearance of a solid mass.
EXHIBIT E: CAD View of Pessary with Hollowed-Out Cylindrical Core
CAD views to illustrate the pessary the hollowed-out core that is roughly cylindrical, to enable the passage of vaginal fluids through this core and/or lighten the invention.

We claim:

1) An intra-vaginal pessary device, that comprises of molded, medical-grade silicone; that is non-absorbent, roughly oval, with smoothed edges and an embedded pull-string for removal from the vagina, having the utility to manage mild to moderate stress incontinence in women by providing non-surgical, and non-invasive, mid-urethral support as a result of pressure against the vaginal walls and urethral opening.

2) An intra-vaginal pessary device, as defined in claim 1, that overcomes the disadvantages of the prior art by being easy to use and fit, self-administered, and therefore capable of being distributed directly to women via retail channels (“off the shelf”), rather than through a physician's office.

3) An intra-vaginal pessary device, as defined in claim 1, that embodies basic, essential product variations to accommodate the needs and differences between women potentially purchasing the invention “off-the-shelf” or at the point of sale. For example, varying sizes to accommodate anatomical differences between women; having the ability to hollow out the core of the pessary to either accommodate those women who have had relatively greater stress to the pelvic floor musculature or to support variations in vaginal dryness between users. The hollowed-out core is roughly cylindrical in shape, lightens the invention and would also allow passage of vaginal fluids if removed.
